(19) United States
(12) Patent Application Publication
(10) Pub. No.: US 2019/0356621 A1
(43) Pub. Date: Nov. 21, 2019

(54) ADAPTING SILENCE PERIODS FOR DIGITAL MESSAGING

(71) Applicant: KONINKLIJKE PHILIPS N.V., Eindhoven (NL)

(72) Inventors: MURTAZA BULUT, EINDHOVEN (NL); DENNIS LOMANS, VELDHOVEN (NL)

(21) Appl. No.: 15/981,932

(22) Filed: May 17, 2018

(51) Int. Cl. H04L 12/58; G06F 17/30; A61B 5/00

(52) U.S. Cl. CPC H04L 51/12; A61B 5/02; A61B 5/0002; G06F 17/30705

(57) ABSTRACT

In one embodiment, an apparatus, comprising: a memory comprising instructions; and one or more processors configured to execute the instructions to: classify a message at least based on an importance of the message; define one or more silence periods based on the classification, the one or more silence periods comprising at least a pre-silence period or a post-silence period, the message adjacent in time to the one or more silence periods; and delay a function involving the message based on the defined one or more silence periods.

### FIELD OF THE INVENTION

The present invention is generally related to digital messaging, and in particular, digital messaging for personal health applications.

### BACKGROUND OF THE INVENTION

Personal health applications use electronics devices, and typically portable electronics devices including wearable devices and/or smartphones, to provide for monitoring and/or rendering consultation to users on continual basis. For instance, a personal health application may deliver digital messages to the user via a phone or wearable interface that serves to inform of progress towards a goal and even influence behavior towards achieving that goal. Messages may be provided via personal apps running on the electronics device, or pushed from a remote server in communications with the electronics device. In either case, one objective is for the messages to actually be opened and reviewed by the user to enable the personal health application to help the user improve his or her health and/or well-being.

One illustration of a personal health application involves coaching applications, where an electronics device in possession of the user may monitor and/or receive data pertaining to physical activity and possible contextual information, and deliver digital messages to the user to assist the user in achieving a particular health goal based on the monitored progress, including losing weight, improving strength, and/or other health benefits. In a coaching program, one desire is that the user always pays attention to the coaching messages. However, making the user pay greater attention to the coaching messages is a challenge, particularly when the user is bombarded with many messages.

### SUMMARY OF THE INVENTION

In one embodiment, an apparatus, comprising: a memory comprising instructions; and one or more processors configured to execute the instructions to: classify a message at least based on an importance of the message; define one or more silence periods based on the classification, the one or more silence periods comprising at least a pre-silence period or a post-silence period, the message adjacent in time to the one or more silence periods; and delay a function involving the message based on the defined one or more silence periods.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which an adaptive messaging system is used, in accordance with an embodiment of the invention.

FIG. 2 is a block diagram that illustrates an example wearable device that may implement all or a portion of the functionality of an adaptive messaging system, in accordance with an embodiment of the invention.

FIG. 3 is a schematic diagram that illustrates an example electronics device that may implement all or a portion of the functionality of an adaptive messaging system, in accordance with an embodiment of the invention.

FIG. 4 is a block diagram that illustrates an example computing device that may implement all or a portion of the functionality of an adaptive messaging system, in accordance with an embodiment of the invention.

FIG. 5 is a block diagram that illustrates an example software architecture for implementing functionality of an adaptive messaging system, in accordance with an embodiment of the invention.

FIG. 6 is a schematic diagram that illustrates example pre- and post-silence periods with durations that are based on an importance of the corresponding message, in accordance with an embodiment of the invention.

FIGS. 7A-7C are schematic diagrams that illustrate example scheduling of silence periods based on an importance of the messages, in accordance with an embodiment of the invention.

FIG. 8 is a flow diagram that illustrates an example adaptive messaging method, in accordance with an embodiment of the invention.

### DETAILED DESCRIPTION OF EMBODIMENTS

Disclosed herein are certain embodiments of an adaptive messaging system and method (collectively hereinafter referred to as an adaptive messaging system) that introduce one or more silent periods in conjunction with the delivery of each digital message for a health application, such as a (digital) coaching program. In one embodiment, each digital coaching message has a pre- and/or post-silence period, and a length (duration) of each silence period is linked to one or more features (e.g. importance, priority, content, type, etc.) of the coaching message. In some embodiments, the length of each silence period may additionally, or alternatively, be linked to the user and/or environmental conditions.

Digressing briefly, in a digital coaching program, one objective is that a user always pays attention to the coaching messages. In an ideal case, the user pays enough attention to all of the messages that are received. However, practically speaking, this is not the case. For instance, based on internal testing and literature results, it is observed that users of coaching programs do not pay attention to all messages that they receive. Moreover, many of the coaching messages delivered to the users' devices are not even opened and viewed. Hence, making users pay greater attention to the coaching messages is a challenge. In contrast, certain embodiments of an adaptive messaging system operate under a premise, indeed a recognition, that silence is a powerful communication tool. In the spoken language, silence can be used to emphasize the preceding or following statement. A good speaker is one that uses silence as a powerful communication tool. For instance, pausing after a powerful statement gives a chance to listeners to better process and reflect on what they had just heard. In addition, silence creates anticipation of what may come next, which naturally may make users more attentive to the following statement. Silence has also been used by creators of music. Powerful songs have a good balance between silence and music. When silence is introduced at the right moments, it can be a very powerful tool. By making clever use of silence, certain embodiments of an adaptive messaging system enable coaching messages to be emphasized, which can lead users paying more attention to the messages, potentially improving an influence of the program and leading to longer usage and stronger bonding. In other words, whereas prior computing devices implementing digital coaching programs have no content-dependent silence periods interspersed between consecutive messages, the invention provides computing devices that provide silence periods that are linked to one or more features of the messaging, which helps to overcome information overload or fatigue common in today's computing devices and hence promote user adherence to messaging and facilitate compliance to the underlying program.

Having summarized certain features of an adaptive messaging system of the present disclosure, reference will now be made in detail to the description of an adaptive messaging system as illustrated in the drawings. While an adaptive messaging system will be described in connection with these drawings, there is no intent to limit it to the embodiment or embodiments disclosed herein. For instance, though emphasis is placed on a digital coaching program as an example health application, it should be appreciated that some embodiments of an adaptive messaging system may be used for other messaging applications that communicate with human users. For instance, certain embodiments of an adaptive messaging system may be beneficially deployed in applications directed to such diverse user groups and/or content as the elderly, children or chronic disease. Further, although the description identifies or describes specifics of one or more embodiments, such specifics are not necessarily part of every embodiment, nor are all of any various stated advantages necessarily associated with a single embodiment. On the contrary, the intent is to cover all alternatives, modifications and equivalents included within the principles and scope of the disclosure as defined by the appended claims. Further, it should be appreciated in the context of the present disclosure that the claims are not necessarily limited to the particular embodiments set out in the description.

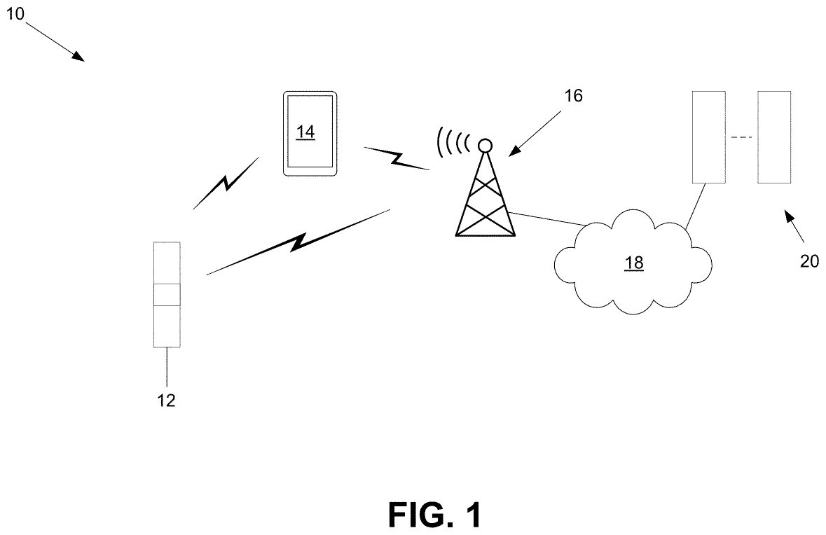

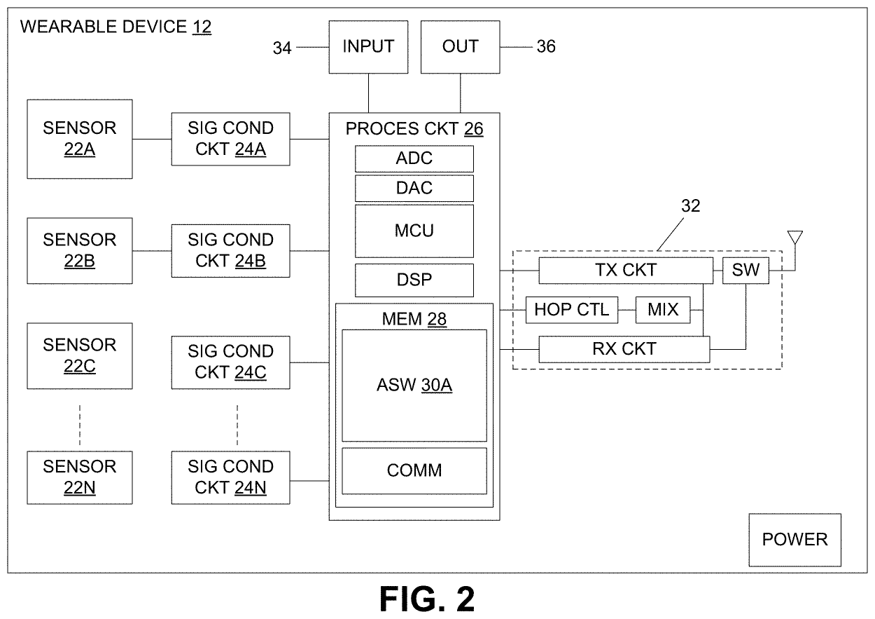

Referring now to FIG. 1, shown is an example environment 10 in which certain embodiments of an adaptive messaging system may be implemented. It should be appreciated by one having ordinary skill in the art in the context of the present disclosure that the environment 10 is one example among many, and that some embodiments of an adaptive messaging system may be used in environments with fewer, greater, and/or different components than those depicted in FIG. 1. The environment 10 comprises a plurality of devices that enable communication of information throughout one or more networks. The depicted environment 10 comprises a wearable device 12, one or more electronics devices 14 (one shown), a wireless/cellular network 16, a wide area network 18 (e.g., also described herein as the Internet, but also including, in some embodiments, one or more of an Internet of Things (IoT) network, an ambient intelligence network, among others), and a remote computing system 20. The wearable device 12, as described further in association with FIG. 2, and in one embodiment, is configured to perform all or at least a portion of the functionality of an adaptive messaging system. The wearable device 12, as described further in association with FIG. 2, is typically worn by the user (e.g., around the wrist, torso, or worn as a patch, or attached to an article of clothing), though some embodiments may embed the device 12 in the body (e.g., underneath the skin). In some embodiments, functionality of the wearable device 12 may be implemented in other types of devices, including house-based devices (e.g., channel control remotes, home smart-speaker assistants, etc.), autonomous vehicles/vehicle components (e.g., the steering wheel, stick, etc.), radios, alarm clocks, etc. In some embodiments, the wearable device 12 may include Google® glasses, wearable lenses, etc. using real time image capture, virtual, or augmented reality. The wearable device 12 comprises a plurality of sensors that track motion (e.g., steps, swim strokes, pedaling strokes, etc.) and/or physical activity (e.g., running, playing football, shopping, riding a bike, etc.) of the user, and sense/measure or derive physiological parameters (e.g., heart rate, respiration, skin temperature, etc.) based on the sensor data, and optionally sense various other parameters (e.g., contextual information, including outdoor temperature, humidity, location, etc.) pertaining to the surrounding environment of the wearable device 12. For instance, in some embodiments, the wearable device 12 may comprise a global navigation satellite system (GNSS) receiver, including a GPS receiver, which tracks and provides location coordinates for the device 12. In some embodiments, the wearable device 12 may comprise indoor location technology, including beacons, RFID or other coded light technologies, WiFi, etc., or other position tracking technology (e.g., using triangulation). Some embodiments of the wearable device 12 may include a motion or inertial tracking sensor, including an accelerometer and/or a gyroscope, providing movement data of the user. A representation of such gathered data may be communicated to the user via an integrated display on the wearable device 12 and/or on another device or devices.

Also, or alternatively, such data collected by the wearable device 12 may be communicated (e.g., continually, periodically, and/or aperiodically, including upon request) via a communications interface to one or more other devices, such as the electronics device 14 or (e.g., via the wireless/cellular network 16) to the computing system 20. Such communications may be achieved wirelessly (e.g., using near field communications (NFC) functionality, Blue-tooth functionality, 802.11-based technology, streaming technology, including LoRa, and/or broadband technology including 3G, 4G, 5G, etc.) and/or according to a wired medium (e.g., universal serial bus (USB), etc.). In some embodiments, the communications interface of the wearable device 12 may receive input from one or more devices, including the electronics device 14 and/or a device(s) of the computing system 20. Further discussion of the wearable device 12 is described below in association with FIG. 2.

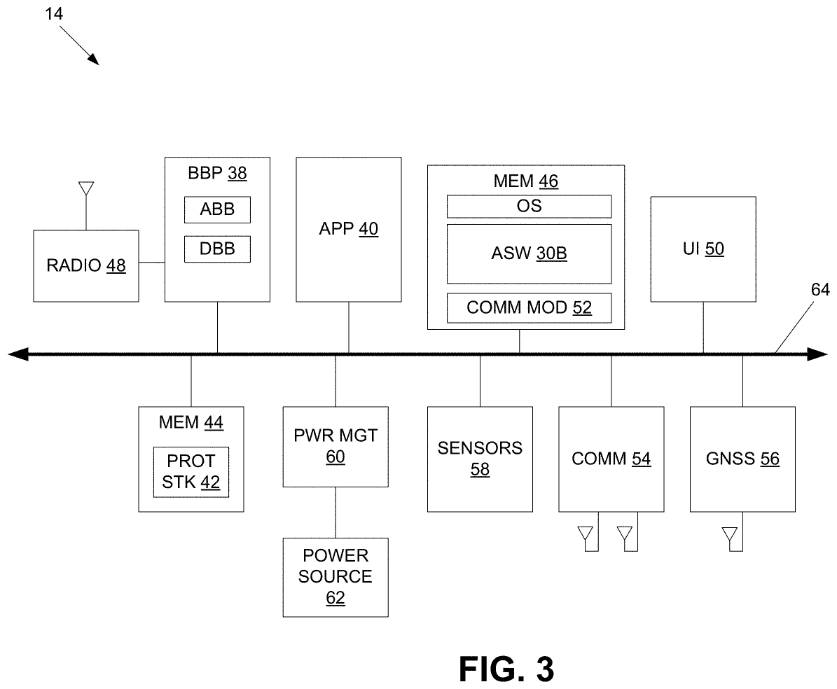

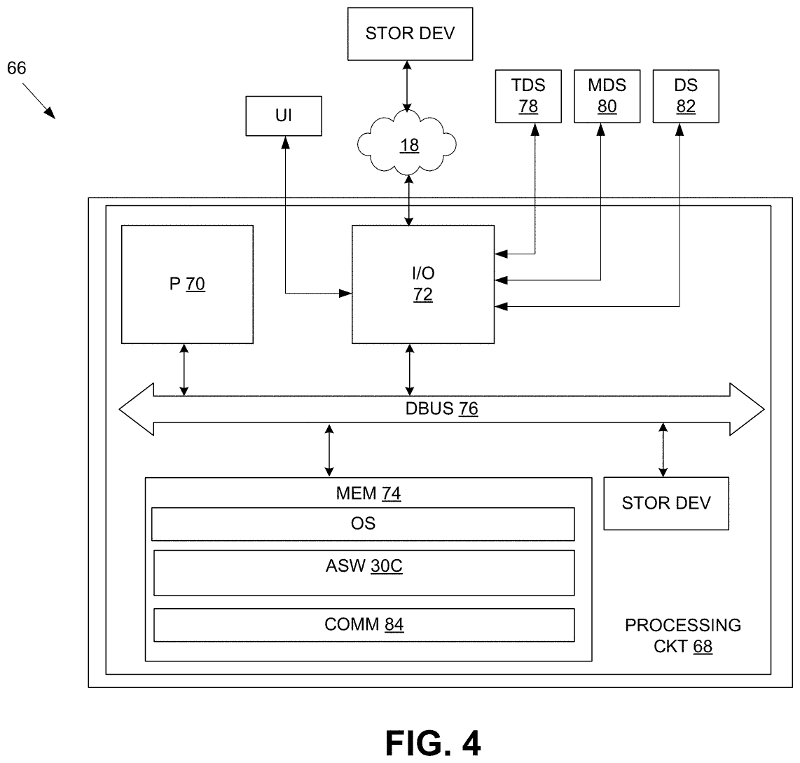

The electronics device 14 may be embodied as a smartphone, mobile phone, cellular phone, pager, stand-alone image capture device (e.g., camera), laptop, personal computer, workstation, among other handheld, portable, or other computing/communication devices, including communication devices having wireless communication capability, including telephony functionality. In the depicted embodiment of FIG. 1, the electronics device 14 is illustrated as a smartphone for convenience in illustration and description, though it should be appreciated that the electronics device 14 may take the form of other types of devices as explained above, including appliances (e.g., implementing the Internet of Things (IoT)), household devices, autonomous vehicles/vehicle components, etc. Further discussion of the electronics device 14 is described below in association with FIG. 3, with the terms smartphone and electronics device 14 used interchangeably hereinafter. Note that the architecture of a personal computer, laptop, workstation, etc. for the electronics device 14 may be similar to that described in connection with FIG. 4 in some embodiments. In one embodiment, the electronics device 14 is configured to perform all or at least a portion of an adaptive messaging system. The electronics device 14 may be in communications with the wearable device 12 and/or one or more devices of the computing system 20, or the same or other types of electronics devices (e.g., smartphones, laptops, etc.). The electronics device 14 may include sensing functionality, including motion (e.g., acceleration) and/or physiological sensing. In one embodiment, the electronics device 14 comprises heart and/or breathing rate monitoring using a Philips Vital Signs Camera, or devices from other manufacturers with similar sensing functionality, to remotely measure heart and breathing rates using a standard, infrared (IR) based camera by sensing changes in skin color and body movement (e.g., chest movement)), among others. The electronics device 14 may further include one or more interfaces for providing digital messaging, including a touch-type display screen. Wireless communication functionality, including cellular, streaming and/or broadband (e.g., 3G, 4G, 5G, LoRa, etc.), Wi-Fi, Blue-tooth, NFC, etc., may be used for the communication of sensing data and/or digital messages among the devices 12, 14, and device(s) of the remote computing system 20), as explained further below in association with FIG. 3.

The wireless/cellular network 16 may include the necessary infrastructure to enable wireless and/or cellular communications between the wearable device 12, the electronics device 14, and one or more devices of the remote computing system 20. There are a number of different digital cellular technologies suitable for use in the wireless/cellular network 16, including: 3G, 4G, 5G, GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others, as well as Wireless-Fidelity (Wi-Fi), 802.11, streaming, etc., for some example wireless technologies.

The wide area network 18 may comprise one or a plurality of networks that in whole or in part comprise the Internet. The wearable device 12 and/or the electronics device 14 may access one or more of the devices of the computing system 20 via the wireless/cellular network 16 and/or the Internet 18, which may be further enabled through access to one or more networks including PSTN (Public Switched Telephone Networks), POTS, Integrated Services Digital Network (ISDN), Ethernet, Fiber, DSL/ADSL, Wi-Fi, among others. For wireless implementations, the cellular/wireless network 16 may use wireless fidelity (Wi-Fi) to receive data converted by the wearable device 12 and/or the electronics device 14 to a radio format and format for communication over the Internet 18. The cellular/wireless network 16 may comprise suitable equipment that includes a modern, router, etc.

The computing system 20 comprises one or more devices coupled to the wide area network 18, including one or more computing devices networked together, including an application server(s) and data storage. The computing system 20 may serve as a cloud computing environment (or other server network) for the wearable device 12 and/or the electronics device 14, performing processing and/or data storage on behalf of (or in some embodiments, in addition to) the wearable device 12 and/or the electronics device 14. One or more devices of the computing system 20 may implement all or at least a portion of certain embodiments of an adaptive messaging system. In one embodiment, the computing system 20 may be configured to be a backend server for a health program. The computing system 20 receives observations (e.g., data) collected via sensors or input interfaces of one or more of the wearable device 12 or electronics device 14 and/or other devices or applications (e.g., third party internet services that provide, for instance, weather reports/forecasts to enable intelligent decisions on whether to recommend an outdoor activity, location services (e.g., Google maps) that provide geospatial data to be used in combination with the received location information (e.g., GPS data) for ascertaining environmental information (e.g., presence of sidewalks), stores the received data in a data structure (e.g., user profile database, etc.), and generates digital messages, including notifications or signals to activate haptic, light-emitting, or aural-based devices or hardware components, among other actions) for presentation to the user. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities, heart rate, respiration rate, blood pressure, body temperature, light and visual information, etc., user feedback/input, and the corresponding context. Based on the data observed during a period of time and/or over a large population of users, the computing system 20 may generate messages pertaining to each specific parameter or a combination of parameters, and provide the messages via the networks 16 and/or 18 for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server or a dedicated computing server. The computing system 20 may be configured to provide backend support for a program developed by a specific manufacturer.

When embodied as a cloud service or services, the device(s) of the remote computing system 20 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the devices of the remote computing system 20 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the devices of the remote computing system 20 may be configured into multiple, logically-grouped servers (run on server devices), referred to as a server farm. The devices of the remote computing system 20 may be geographically dispersed, administered as a single entity, or distributed among a plurality of server farms, executing one or more applications on behalf of, or processing data from, one or more of the wearable device 12 and/or the electronics device 14. The devices of the remote computing system 20 within each farm may be heterogeneous. One or more of the devices may operate according to one type of operating system platform (e.g., WINDOWS NT, manufactured by Microsoft Corp. of Redmond, Wash.), while one or more of the other devices may operate according to another type of operating system platform (e.g., Unix or Linux). The group of devices of the remote computing system 20 may be logically grouped as a farm that may be interconnected using a wide-area network (WAN) connection or medium-area network (MAN) connection. The devices of the remote computing system 20 may each be referred to as, and operate according to, a file server device, application server device, web server device, proxy server device, or gateway server device.

In one embodiment, the computing system 20 may comprise a web server that provides a web site that can be used by users to review information related to monitored behavior/activity and/or review/update user data and/or a record of measurements. The computing system 20 may receive data collected via one or more of the wearable device 12 and/or the electronics device 14, store the received data in a data structure (e.g., user profile database) along with one or more tags, process the information (e.g., to determine an appropriate message), and deliver the message at one or more of the devices 12 and/or 14. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. In one embodiment, the data received at the computing system 20 may be stored in a user profile data structure comprising a plurality of measurements pertaining to activity/inactivity, for example, body movements, sensed physiological measurements, including heart rate (e.g., average heart rate, heart rate variations), heart rhythm, inter-beat interval, respiration rate, blood pressure, body temperature, etc., context (e.g., location, environmental conditions, etc. tagged to one or more of the measurements), and/or a history of feedback messages. In some embodiments, the computing system 20 is configured to be a backend server for a health-related program or a health-related application implemented on the wearable device 12 and/or the electronics device 14. The functions of the computing system 20 described above are for illustrative purpose only. The present disclosure is not intended to be limiting. The computing system 20 may be a general computing server device or a dedicated computing server device. The computing system 20 may be configured to provide backend support for a program developed by a specific manufacturer. However, the computing system 20 may also be configured to be interoperable across other server devices and generate information in a format that is compatible with other programs. In some embodiments, one or more of the functionality of the computing system 20 may be performed at the respective devices 12 and/or 14.

Note that cooperation between the wearable device 12 and/or the electronics device 14 and the one or more devices of the computing system 20 may be facilitated (or enabled) through the use of one or more application programming interfaces (APIs) that may define one or more parameters that are passed between a calling application and other software code such as an operating system, library routine, function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer employs to access functions supporting the API. In some implementations, an API call may report to an application the capabilities of a device running the application, including input capability, output capability, processing capability, power capability, and communications capability. Further discussion of an example device of the computing system 20 is described below in association with FIG. 4.

An embodiment of an adaptive messaging system may comprise the wearable device 12, the electronics device 14, and/or the computing system 20. In other words, one or more of the aforementioned devices 12, 14, and device(s) of the remote computing system 20 may implement the functionality of the adaptive messaging system. For instance, the wearable device 12 may comprise all of the functionality of an adaptive messaging system, enabling the user to avoid or limit the need for Internet connectivity and/or any inconvenience in carrying a smartphone 14 around. In some embodiments, the functionality of an adaptive messaging system may be implemented using any combination of the wearable device 12, the electronics device 14, and/or the computing system 20. For instance, the wearable device 12 may provide for sensing functionality and a rudimentary feedback capability, the electronics device 14 may provide a more sophisticated interface for the presentation of messages, monitoring functionality for when messages are opened and/or read by the user, and serve as a communications intermediary between the computing system 20 and the wearable device 12, and the computing system 20 may receive (e.g., from the wearable device 12 and/or the smartphone 14) the measurement and/or contextual data (and possibly indications of when a user opens messages) from the devices 12, 14 and responsively provide messages (e.g., coaching messages) to the electronics device 14 for presentation. These and/or other variations, including distributed processing, measurement, etc., may be used among the devices/system 12, 14, and/or 20, and hence are contemplated to be within the scope of the disclosure.

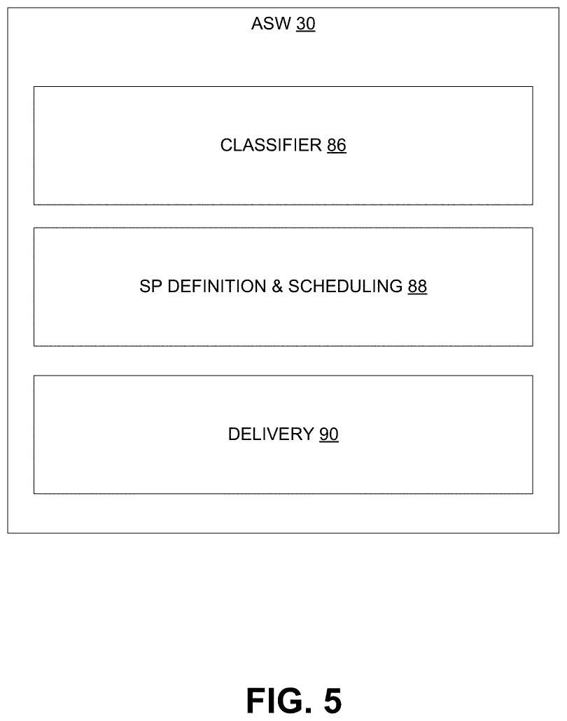

Having generally described an example environment 10 in which an embodiment of an adaptive messaging system may be implemented, attention is directed to FIG. 2. FIG. 2 illustrates example circuitry for the example wearable device 12, and in particular, underlying circuitry and software (e.g., architecture) of the wearable device 12 that in one embodiment is used to implement an adaptive messaging system. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist exclusively or primarily of the microcontroller. In some embodiments, the processing circuit 26 may include the signal conditioning circuits 24. The memory 28 comprises an operating system (OS) and application software (ASW) 30A. The application software 30A comprises instructions/executable code to implement all or a portion of the adaptive messaging system, as is described further below in conjunction with FIG. 5. The memory 28 also comprises communications software (COMM), such as that used to enable the wearable device 12 to operate according to one or more of a plurality of different communication technologies (e.g., GSM, WCDMA, 3G, 4G, 5G, streaming (e.g., LoRa), NFC, Bluetooth, Wi-Fi, Zigbee, etc.). In some embodiments, the communications software may be part of the application software 30A or located in separate or other memory.

In one embodiment, the processing circuit 26 is coupled to a communications circuit 32. The communications circuit 32 serves to enable wireless communications between the wearable device 12 and other devices, including the electronics device 14 and the computing system 20, among other devices. The communications circuit 32 is depicted as a Bluetooth circuit, though not limited to this transceiver configuration. For instance, in some embodiments, the communications circuit 32 may be embodied as one or any combination of an NFC circuit, Wi-Fi circuit, transceiver circuitry based on Zigbee, 802.11, GSM, LTE, CDMA, WCDMA, circuitry for enabling broadband and/or streaming (e.g., 3G, 4G, 5G, LoRa, etc.), among others such as optical or ultrasonic based technologies. The processing circuit 26 is further coupled to input/output (I/O) devices or peripherals, including an input interface 34 (INPUT) and an output interface 36 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 perform detection and measurement of a plurality of physiological and behavioral parameters (e.g., typical behavioral parameters or activities including walking, running, cycling, and/or other activities, including shopping, walking a dog, working in the garden, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body fat percentage, etc.). At least one of the sensors 22 may be embodied as movement detecting sensors, including inertial sensors (e.g., gyroscopes, single or multi-axis accelerometers, such as those using piezoelectric, piezoresistive or capacitive technology in a microelectromechanical system (MEMS) infrastructure for sensing movement) and/or as GNSS sensors, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., WiFi, coded-light based technology, acoustic-based tracking, etc.) and/or other tracking/location mechanisms. The sensors 22 may also include flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. The sensors 22 may include other and/or additional types of sensors for the detection of, for instance, environmental conditions including barometric pressure, humidity, outdoor temperature, etc. In some embodiments, GNSS functionality may be achieved via the communications circuit 32 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22) or fewer sensors 22 may be used. In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 32 is managed and controlled by the processing circuit 26 (e.g., executing the communications software), though in some embodiments, the communications circuit 32 may be implemented without software control. The communications circuit 32 is used to wirelessly interface with the electronics device 14 (FIG. 3) and/or one or more devices of the computing system 20 (FIG. 4). In one embodiment, the communications circuit 32 may be configured as a Bluetooth (including BTLE) transceiver, though in some embodiments, other and/or additional technologies may be used, such as Wi-Fi, 3G, 4G, 5G, GSM, LTE, CDMA and its derivatives, Zigbee, NFC, streaming, among others. In the embodiment depicted in FIG. 2, the communications circuit 32 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 32 may be implemented by the microcontroller, the DSP, or a combination of both. In some embodiments, the communications circuit 32 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software, which when executed by the microcontroller, controls the Bluetooth (and/or other protocols) transmission/reception.

Though the communications circuit 32 is depicted as an IF-type transceiver, in some embodiments, a direct conversion architecture may be implemented. As noted above, the communications circuit 32 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 34 to a digital format for further processing by the microcontroller. The ADC may also be used in baseband processing of signals received via the communications circuit 32. The DAC converts digital information to analog information. Its role for sensing functionality may be to control the emission of signals, such as optical signals or acoustic signal, from the sensors 22. The DAC may further be used to cause the output of analog signals from the output interface 36. Also, the DAC may be used to convert the digital information and/or instructions from the microcontroller and/or DSP to analog signals that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to, say, a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters and/or location coordinates or other contextual information based on the sensors 22, and for enabling communication with the electronics device 14 and/or a device of the computing system 20, and in some embodiments, for the presentation of messages.

The memory 28 can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30A, which may run (or work in conjunction with) a health program and further includes one or more algorithms for determining physiological and/or behavioral measures and/or other information (e.g., including location, speed of travel, environmental, etc.) based on the output from the sensors 22. The raw data from the sensors 22 may be used by the algorithms to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12. In some embodiments, the GPS functionality of the sensors 22 collects contextual data (time and location data, including location coordinates). The application software 30A may also collect information about the means of ambulation. For instance, the GPS data (which may include time coordinates) may be used by the application software 30A to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data. In some embodiments, the application software 30A further comprises software to provide messages (e.g., generated at the wearable device 12 or at another device(s), including the electronics device 14 and/or a device of the computing system 20). In some embodiments, the application software 30A derives one or more silence periods to be used in conjunction with each message, though in some embodiments, the application software 30A receives commands for the implementation of the silence periods from other devices or the messages with the silence periods from other devices.

The operating system essentially controls the execution of other computer programs, such as the application software 30A, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts (e.g., environmental conditions, user state, etc.), and/or in some embodiments, past messages (e.g., including type of message, format, frequency of delivery, message distribution, delivery channel(s), times of delivery, associated cards used for the delivery mechanism and respective features, use of the message (e.g., whether links were selected, read, etc.), past responses to messages, past silence periods, etc.). In some embodiments, the memory 28 may store other data, including information about the status of the network, the periods the network used for communication was down or working properly, signal strength, among other parameters related to the medium of communication and/or the signals. In some embodiments, one or more of the historical data and/or other information may be stored at one or more other devices. In some embodiments, the application software 30A may comprise learning algorithms, data mining functionality, among other features. For instance, if there exists no messages for an extended period of time, there is a strong likelihood that the current message is important (e.g., in terms of how it is perceived by the user and how it is expected to influence the user). In some embodiments, similar functionality may reside at another device.

Although the application software 30A is described above as being implemented in the wearable device 12, some embodiments may distribute the corresponding functionality among the wearable device 12 and other devices (e.g., electronics device 14 and/or one or more devices of the computing system 20 and/or a vehicle), or in some embodiments, the application software 30A may be implemented in another device (e.g., the electronics device 14, a device of the computing system 20).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 34 comprises an interface (e.g., including a user interface) for entry of user input, such as a button or microphone or sensor (e.g., to detect user input, including gestures, physiological signals, etc.) or touch-type display. In some embodiments, the input interface 34 may serve as a communications port for downloaded information to the wearable device 12 (such as via a wired connection). The output interfaces 36 comprises an interface for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 36 may be used to present messages to the user. In some embodiments, at least some of the functionality of the input and output interfaces 34 and 36, respectively, may be combined, including being embodied at least in part as a touch-type display screen for the entry of input (including replies to questionnaires) and to provide digital messages (e.g., coaching messages). The wearable device 12 may also include a power source (POWER), such as a battery.

Referring now to FIG. 3, shown is an example electronics device 14 in which all or a portion of the functionality of an adaptive messaging system may be implemented. In the depicted example, the electronics device 14 is embodied as a smartphone (hereinafter, referred to as smartphone 14 for illustration and convenience), though in some embodiments, other types of devices may be used, such as a workstation, laptop, notebook, tablet, home appliance, vehicle/vehicle component, etc. It should be appreciated by one having ordinary skill in the art that the logical block diagram depicted in FIG. 3 and described below is one example, and that other designs may be used in some embodiments. The smartphone 14 comprises application software (ASW) 30B, which may include all or a portion of the functionality of an adaptive messaging system. Functionality of certain embodiments of an adaptive messaging system may be carried out entirely using the smartphone 14, or in some embodiments, carried out in part with the cooperation of additional devices of the environment 10 (FIG. 1). The smartphone 14 comprises at least two different processors, including a baseband processor (BBP) 38 and an application processor (APP) 40. As is known, the baseband processor 38 primarily handles baseband communication-related tasks and the application processor 40 generally handles inputs and outputs and all applications other than those directly related to baseband processing. The baseband processor 38 comprises a dedicated processor for deploying functionality associated with a protocol stack (PROT STK) 42, such as a GSM (Global System for Mobile communications) protocol stack, among other functions. The application processor 40 comprises a multi-core processor for running applications, including all or a portion of the application software 30B. The baseband processor 38 and application processor 40 have respective associated memory (e.g., MEM) 44, 46, including random access memory (RAM), Flash memory, etc., and peripherals, and a running clock. Note that, though depicted as residing in memory 46, all or a portion of the application software 30B may be stored in memory 44, distributed among memory 44, 46, or reside in other memory.

More particularly, the baseband processor 38 may deploy functionality of the protocol stack 42 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), broadband (e.g., 3G, 4G, 5G), streaming services (e.g., LoRa), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 38 manages radio communications and control functions, including signal modulation, radio frequency shifting, and encoding. The baseband processor 38 comprises, or may be coupled to, a radio (e.g., RF front end) 48 and/or a GSM modem, and analog and digital baseband circuitry (ABB, DBB, respectively in FIG. 3). The radio 48 comprises one or more antennas, a transceiver, and a power amplifier to enable the receiving and transmitting of signals of a plurality of different frequencies, enabling access to the wireless/cellular network 16 (FIG. 1), and hence sending or receiving communications involving user data, measurements, associated contexts, and/or messages and silence periods. The analog baseband circuitry is coupled to the radio 48 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 40 or directly from the smartphone user interface (UI) 50 (e.g., microphone, speaker, earpiece, ring tone, vibrator circuits, display screen, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 40). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 40 operates under control of an operating system (OS) that enables the implementation of one or a plurality of user applications, including the application software 30B and a health or coaching application. The application processor 40 may be embodied as a System on a Chip (SOC), and supports a plurality of multimedia related features including web browsing functionality to access one or more computing devices of the computing system 20 (FIG. 1) that are coupled to the Internet, email, multimedia entertainment, games, etc. For instance, the application processor 40 may execute communications module 52, which may include middleware (e.g., browser with or operable in association with one or more application program interfaces (APIs)) to enable access to a cloud computing framework or other networks to provide remote data access/storage/processing, and through cooperation with an embedded operating system, access to calendars, location services, reminders, etc. The application processor 40 generally comprises a processor core (Advanced RISC Machine or ARM), and further comprises or may be coupled to multimedia modules (for decoding/encoding pictures, video, and/or audio), a graphics processing unit (GPU), communications interface (COMM) 54, and device interfaces. In one embodiment, the communication interfaces 54 may include wireless interfaces, including a Bluetooth (BT) (and/or Zigbee in some embodiments) module, 3G, 4G, or 5G module, streaming module, etc. that enable wireless communications with one or more devices of the environment 10 (FIG. 1), including the wearable device 12, and/or a Wi-Fi module for interfacing with a local 802.11 network, according to corresponding software in the communications module 52. The application processor 40 further comprises, or is coupled to, a global navigation satellite systems (GNSS) transceiver or receiver (GNSS) 56 for enabling access to a satellite network to, for instance, provide coordinate location services. In some embodiments, the GNSS receiver 56, in association with GNSS functionality in the application software 30B (e.g., as part of position determining software or integrated in the communications module 52), collects contextual data (time and location data, including location coordinates and altitude), which may be used for storage with measured data. In some embodiments, other and/or additional location technology may be used, including location through triangulation techniques.

In one embodiment, the smartphone comprises sensors 58, which may include one or any combination of a PPG sensor or a motion sensor(s) (e.g., an accelerometer, inertial sensors, including a gyroscope). For instance, the PPG sensor may be embodied as an optical sensor (e.g., a charged coupled device (CCD) or a complementary metal-oxide semiconductor (CMOS) optical sensor), which is used to detect various physiological parameters of a user, including blood pressure or breathing rate based on remote photoplethysmography (PPG). The sensors 58 may also include other types of sensors, including electromyograph (EMG) sensors, impedance sensors, skin temperature sensors, environmental sensors, etc.

The device interfaces coupled to the application processor 40 may include the user interface 50, including a display screen. The display screen, which may be similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the application software 30B may cause the rendering on the UI 50 of web pages, dashboards, and/or feedback (e.g., messages). Other and/or additional user interfaces 50 may include a keypad, microphone, speaker (e.g., to audibly present messages), ear piece connector, I/O interfaces (e.g., USB (Universal Serial Bus)), SD/MMC card, lighting (e.g., to provide a visualized feedback, including via different colored LEDs or different illumination patterns of the LEDs), or a tactile device (e.g., vibration motor to provide tactile feedback), among other peripherals.

Also included is a power management device 60 that controls and manages operations of a power source (e.g., battery) 62. The components described above and/or depicted in FIG. 3 share data over one or more busses, and in the depicted example, via data bus 64. It should be appreciated by one having ordinary skill in the art, in the context of the present disclosure, that variations to the above example description of the smartphone 14 may be deployed in some embodiments to achieve similar functionality.

In the depicted embodiment, the application processor 40 runs the application software 30B, which in one embodiment, includes a plurality of software modules (e.g., executable code/instructions) to carry out all or a portion of the functionality of an adaptive messaging system. Further description of the application software 30B (and 30A, FIG. 2) is described in association with FIG. 5.

The communications module 52 comprises executable code (instructions) to enable the communications interface 54 and/or the radio 48 to communicate with other devices of the environment, including the wearable device 12 and/or one or more devices of the computing system 20 and/or other devices. Communications may be achieved according to one or more communications technologies, including 3G, 4G, 5G, GSM, LTE, CDMA, WCDMA, Wi-Fi, 802.11, Bluetooth, NFC, streaming technology, etc.). The communications module 52 may also include browser software in some embodiments to enable Internet connectivity. The communications module 52 may also be used to access certain services, such as mapping/place location services, which may be used to determine a context for the sensor data.

Having described the underlying hardware and software of the wearable device 12 and the electronics device 14, attention is now directed to FIG. 4, which illustrates an example computing device 66 of the computing system 20, which may be used to implement all or at least a portion of the functionality of an adaptive messaging system. The computing device 66 may be embodied as an application server, computer, among other computing devices, and is also generally referred to herein as an apparatus. One having ordinary skill in the art should appreciate in the context of the present disclosure that the example computing device 66 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing device 66 is depicted in this example as a computer system, such as one providing a function of an application server. It should be appreciated that certain well-known components of computer systems are omitted here to avoid obfuscating relevant features of the computing device 66. In one embodiment, the computing device 66 comprises a processing circuit 68 comprising hardware and software components. In some embodiments, the processing circuit 68 may comprise additional components or fewer components. For instance, memory may be separate. The processing circuit 68 comprises one or more processors, such as processor 70 (P), input/output (I/O) interface(s) 72 (I/O), and memory 74 (MEM), all coupled to one or more data busses, such as data bus 76 (DBUS). The memory 74 may include one or any combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.).

The memory 74 may store a native operating system (OS), one or more native applications, emulation systems, or emulated applications for any of a variety of operating systems and/or emulated hardware platforms, emulated operating systems, etc. In some embodiments, the processing circuit 68 may include, or be coupled to, one or more separate storage devices. For instance, in the depicted embodiment, the processing circuit 68 is coupled via the I/O interfaces 72 to template data structures (TDS) 78 and message data structures (MDS) 80, and further to data structures (DS) 82. Note that in some embodiments, one or more of these data structures 78, 80, 82, or similar with a reduced data set, may be stored at the devices 12 and/or 14. In some embodiments, the template data structures 78, message data structures 80, and/or data structures 82 may be coupled to the processing circuit 68 via the data bus 76 or coupled to the processing circuit 68 via the I/O interfaces 72 as network-connected storage devices. The data structures 78, 80, and/or 82 may be stored in persistent memory (e.g., optical, magnetic, and/or semiconductor memory and associated drives). In some embodiments, the data structures 78, 80, and/or 82 may be stored in memory 74.

The template data structures 78 are configured to store one or more templates that are used in a message definition stage to generate the messages conveying information to the user. A message for different objectives may use different templates. For example, education related messages may apply templates with referral links to educational resources, feedback on performance may apply templates with rating/ranking comments, etc. The template data structures 78 may be maintained by an administrator operating the computing system 20 and/or computing device 66. The template data structures 78 may be updated based on the usage of each template, the feedback on each generated message, among other metrics. The templates that are more often used and/or receive more positive feedbacks from the users may be highly recommended to generate the messages in the future. In some embodiments, the templates may be general templates that can be used to generate all types of messages. In some embodiments, the templates may be classified into categories, each category pertaining to a parameter. For example, templates for generating messages pertaining to heart rate may be partially different from templates for generating messages pertaining to sleep quality. The message data structures 80 are configured to store the messages that are constructed based on the templates. The data structures 82 are configured to store user profile data including the real-time measurements of parameters for a large population of users, personal information of the large population of users, user-entered input, etc. In some embodiments, the data structures 82 are configured to store health-related information of the user and/or contextual data. The data structures 78-82 may be backend databases of the computing system 20 and/or the computing device 66. In some embodiments, however, the data structures 78-82 may be in the form of network storage and/or cloud storage directly connected to the network 18 (FIG. 1). In some embodiments, the data structures data structures 78-82 may serve as backend storage of the computing system 20 as well as network storage and/or cloud storage. The data structures data structures 78-82 are updated periodically, aperiodically, and/or in response to a request from the wearable device 12, the electronics device 14, and/or the operations of the computing system 20 and/or computing device 66. Note that in some embodiments, the data structures 78-82 may be combined into fewer data structures or extended to additional data structures.

In the embodiment depicted in FIG. 4, the memory 74 comprises an operating system (OS) and the application software (ASW) 30C, which is described further below in association with FIG. 5. In some embodiments, the operating system may be omitted. The memory 74 further comprises a communications module (COMM) 84 that, in cooperation with the application software 30C, formats the messages to be delivered according to one or any combination of human-perceivable formats (e.g., visually, audibly, using tactile feedback, including Braille, etc.). In one embodiment, the communications module 84, in cooperation with the application software 30C, may comprise card presentation functionality. In some embodiments, card functionality resides in the application software 30C. As used herein, content cards generated for a specific parameter or plural parameters define a family of messages associated with the respective or collective parameters. For example, the content cards generated for sleep quality define a family of messages related to sleep quality, while the content cards generated for running define a family of messages related to running. The content cards may be configured to present one message per card, though in some embodiments, additional messages may be presented. Different families may define a different numbers of messages for presentation. In some embodiments, the content cards may be configured to present respective messages related to the feedback of an activity performance. In some embodiments, the content cards may be configured to present messages comprising educational information, or medication information, etc. In some embodiments, the content cards may be configured to present respective messages comprising insightful analysis of the user's health-related conditions. In some embodiments, the content cards may comprise only text statements. In some embodiments, the content cards may comprise content in multiple formats including but not limited to text, audio, video, flash, hyperlink to other sources, etc. It should be appreciated that the content cards may be generated for purposes other than the examples described above, and the format of the content cards may be adjustable for presentation on different user devices. The examples set forth above are for illustrative purposes, and the present disclosure is not intended to be limiting. For instance, presentation of the messages is not limited to content card formats.

In one embodiment, the communications module 84, in cooperation with the application software 30C, is configured to receive the messages, and prepare the presentation of the content cards based on settings pre-defined by the user and/or the configuration of each individual user device. The settings pre-defined by the user may comprise how the user wants to be notified with the content cards, for example, in a text format, in a chart format, in an audio format with low-tone female voice, in a video/flash format, and/or the combinations thereof. The settings pre-defined by the user may further comprise when and how often the user wants to be notified with the content cards, for example, every evening around 9:00 pm, every afternoon after exercise, every week, every month, in real-time, and/or the combination thereof. The settings pre-defined by the user may further comprise a preferred user device to receive the content card if the user has multiple devices. The configuration of each individual user device may include the size and resolution of the display screen of a user device, the caching space of the user device, etc. In some embodiments, the communications module 84, in cooperation with the application software 30C, may determine the connection status of the user device before sending the content cards. If the user device is determined to be unavailable due to power off, offline, damaged, etc., the communications module 84 may store the generated content card in memory 74 and/or upload the generated content card to the data structures 82. Once the user is detected logged-in using one of his or her user devices, the generated content card is transmitted to the user device for presentation. In some embodiments, if the preferred user device is unavailable, the communications module 84 adjusts the content card for presentation in the logged-in user device. In some embodiments, when to present the content cards may be learned (e.g., using machine learning), such as based on feedback as to positive (or negative) efficacy and/or engagement. Note that the application software 30C may also determine silence periods, which may dictate when the content cards are delivered and/or presented relative to other cards, as explained below in association with FIG. 5.

The communications module 84 further enables communications among network-connected devices and provides web and/or cloud services, among other software such as via one or more APIs. For instance, the communications module 84, in cooperation with the application software 30C, may receive (via I/O interfaces 72) input data (e.g., a content feed) from the wearable device 12 and/or the electronics device 14 that includes sensed data and a context for the sensed data, data from third-party databases (e.g., medical data base, weather data, mapping data), data from social media, data from questionnaires, data from external devices (e.g., weight scales, environmental sensors, etc.), among other data. The content feed may be continual, intermittent, and/or scheduled. The communications module 84 operates in conjunction with the I/O interfaces 72 and the application software 30C to provide the messages to the wearable device 12 and/or the electronics device 14.

Execution of the application software 30C may be implemented by the processor 70 under the management and/or control of the operating system. The processor 70 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 66.

The I/O interfaces 72 comprise hardware and/or software to provide one or more interfaces to the Internet 18, as well as to other devices such as a user interface (UI) (e.g., keyboard, mouse, microphone, display screen, etc.) and/or the data structures 78-82. The user interfaces may include a keyboard, mouse, microphone, immersive head set, display screen, etc., which enable input and/or output by an administrator or other user. The I/O interfaces 72 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing device 66. The administrator may input a request via the user interface, for instance, to manage the template data structure 78. Upon receiving the request, the processor 70 instructs a template building component to process the request and provide information to enable the administrator to create, modify, and/or delete the templates.

When certain embodiments of the computing device 66 are implemented at least in part with software (including firmware), as depicted in FIG. 4, it should be noted that the software can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 66 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Attention is now directed to FIG. 5, which illustrates an example software architecture for the application software (ASW) 30 (e.g., 30A, 30B, 30C), which is used to implement the functionality of an adaptive messaging system. The application software 30 may reside entirely within a single device, including the wearable device 12 (FIG. 2), the electronics device 14 (FIG. 3), or the computing device 66 (FIG. 4), or in some embodiments, the functionality of the application software 30 may be distributed among plural devices (e.g., the wearable device 12, the electronics device 14, and/or the computing device 66). In one embodiment, the application software 30 comprises executable code/instructions comprising a classifier module (CLASSIFIER) 86, a silence period definition and scheduling module (SP DEFINITION & SCHEDULING) 88, and a delivery module (DELIVERY) 90. It should be appreciated by one having ordinary skill in the art, in the context of the present disclosure, that variations to the architecture may be implemented, including fewer and/or additional modules, and hence such variations are contemplated to be within the scope of the disclosure. In some embodiments, functionality corresponding to one or more of the modules 86-90 may be implemented in hardware. For illustrative purposes, and in the interest of brevity, the application software 30 is primarily described below as being executed by the computing device 66 (with the actual presentation of the messages being performed at the wearable device 12 and/or the electronics device 14), with the understanding that processing may be achieved in other devices or among plural devices in some embodiments.

In general, the application software 30 may run, or operate in conjunction with, a health application, including a coaching application. Note that reference to a coaching application for a health application is illustrative of one example implementation, and that in some embodiments, any type of user-engagement application in the fields or endeavors of health, finance, business, etc. may be used where intelligent use of silence periods is effective in improving user engagement with the application. The coaching application triggers a coaching message based on input gathered from the wearable device 12 and/or electronics device 14, including physical activity, behavior, user state, and context, the coaching message intended to influence the user in, for instance, advancing progress towards a goal (e.g., entered by the user, including losing weight, building endurance, etc.). Based on a triggering of the coaching message, the classifier module 86 determines one or more features/parameters of, or associated with, the coaching message based on one or a plurality of sources of input, as explained further below. The classifier module 86 passes these features, or indications of these features, to the silence period definition and scheduling module 88, which defines and schedules one or more silence periods to be configured as a prefix to the message and/or a suffix to the coaching message based on these features/parameters. A silent period or silence period refers to herein as a period when the user will not perceive any coaching messages (e.g., cards) or notifications, after a triggering of the messages by the coaching application, that is deliberate, intentional, variable, and designed or programmed for an intended beneficial effect on influencing behavior. For instance, in one embodiment, the silence period definition and scheduling module 88 determines a time and duration of the silence periods by taking into account an importance of the coaching message and a delivery time of the coaching message, which are features/parameters determined by the classifier module 86. In some embodiments, one or more additional features/parameters may be considered in the definition and scheduling of the coaching message, including a distribution of coaching messages in time, current user state, observed reaction (to previously delivered coaching messages) of the user, a format of the coaching message (e.g., text, audio, etc.), and environmental factors. In embodiments where the silence period definition and scheduling module 88 resides in a local device (e.g., the wearable device 12 and/or the electronics device 14, FIG. 1), the silence periods are introduced in the device. For instance, once a message is received (e.g., from the computing device 66, FIG. 4) and classified (e.g., locally or at the computing device 66), the device (e.g., the silence period definition and scheduling module 88) may operate in what may be referred to as a “do not disturb” mode, where the device prohibits the receipt or display of any other messages for a certain period of time after the received message (or prior to displaying the message) based on the one or more features identified or derived through the classification of the message (e.g., card importance). One benefit of such an embodiment is that the adaptive messaging system can be deployed for multiple independent apps running on the device. Another or alternative benefit to this embodiment is that complete silence can be ensured in the device during this time period (e.g., no messages from social media apps, browser apps, etc.) In some embodiments, functionality of the silence period definition and scheduling module 88 may reside at the network level (e.g., a home network of the Internet of Things), which may prohibit (deny) the passage/delivery of messages from any of the devices of the network during a silence period. Note that one or more of the functionality described above for the silence period definition and scheduling module 88 may instead be performed in another of the modules (e.g., the delivery module 90) in some embodiments, and in some embodiments, one or more of the functionality may be combined or re-distributed among one or more of the modules. The silence period definition and scheduling module 88 also determines when the coaching message is to be presented relative to a silence period of a prior coaching message, establishing priority among the silence periods based on the importance of the current and prior coaching messages. The silence period definition and scheduling module 88 passes this information to the delivery module 90, which delivers the coaching message according to the silence periods determined by the silence period definition and scheduling module 88.

In some embodiments, the duration of each of the silence periods may range from zero to infinity (indefinite), or some range in between, where in some embodiments, there is an opportunity to cancel the delivery of a triggered message. In some embodiments, silence periods may be modified any time before a coaching message is actually delivered (or notification generated, as explained below). In other words, initially the silence periods may be calculated when the coaching message is triggered. However later, if for some reason (e.g., user actions, context, message content, etc.) the application software 30 decides that the silence periods should be altered, then the application software 30 may make these alterations. For instance, one way to implement this modification is to check if the silence periods (e.g., chosen duration values) need to be updated at a certain frequency (e.g., every 30 sec) or whenever new data becomes available. In some embodiments, instead of, or in addition to, a modification to the silence periods, the coaching message may be replaced or changed (e.g., if the coaching message has not been delivered or viewed or perceived by the user), and if the coaching message has changed, calculations of the silence period values are to be updated.

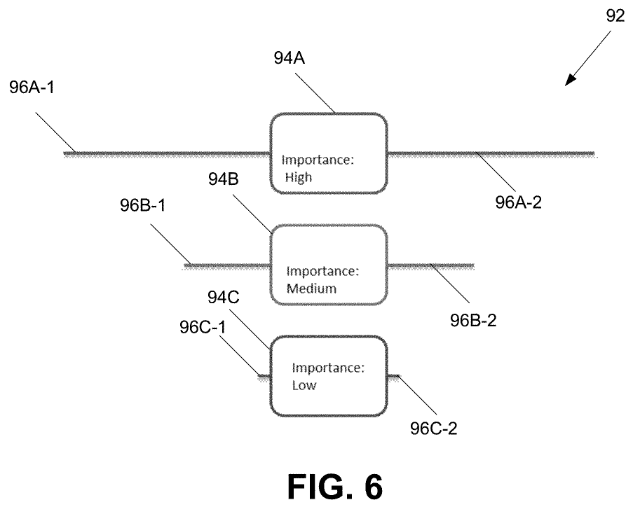

Attention is now directed to FIG. 6 (with continued reference to FIG. 5), which illustrates an embodiment of an adaptive messaging system where silence periods are inserted at both ends of the coaching message, with the understanding that in some embodiments, insertion may only occur before or after a given coaching message. In one embodiment, the silence period definition and scheduling module 88 determines a length/duration of the silence period that is linked to a feature or features of the coaching message, including importance, priority, content, type, context, etc. In the depicted example of FIG. 6, a set 92 of message 94 and respective silence periods 96 are shown, where the length of the silence periods are positively correlated to the importance of the messages. The lines extending from each message 94 indicate the silence periods 96. Each message 94 has a pre- and post-silence period 96 that is linked to the message feature/parameter, such as importance, priority, content, context, etc. In particular, the feature of the message 94 (e.g., as derived from the classifier module 86) used by the silence period definition and scheduling module 88 to determine the length of the silence periods 96A-1, 96A-2, is importance. As shown reviewing the set 92 from top-down, the relative importance of the messages 94 goes from high (94A) to medium (94B) to low (94C), with a corresponding decreasing length for the silence periods 96. For instance, silence periods 96A-1 (pre), 96A-2 (post) have the longest duration because of the highest importance of the message 94A, silence periods 96B-1 (pre) and 96B-2 (post) have respective lengths for the message 94B of medium importance that are shorter than the silence periods for the high importance message 94A, and silence periods 96C-1 (pre) and 96C-2 (post) have respective lengths that are the shortest among the set 92 based on having the lowest importance message 94C. Although the depicted embodiment is shown with a length/duration of the silence period positively correlated to the message importance, some embodiments may use other or additional features to define the silence periods.

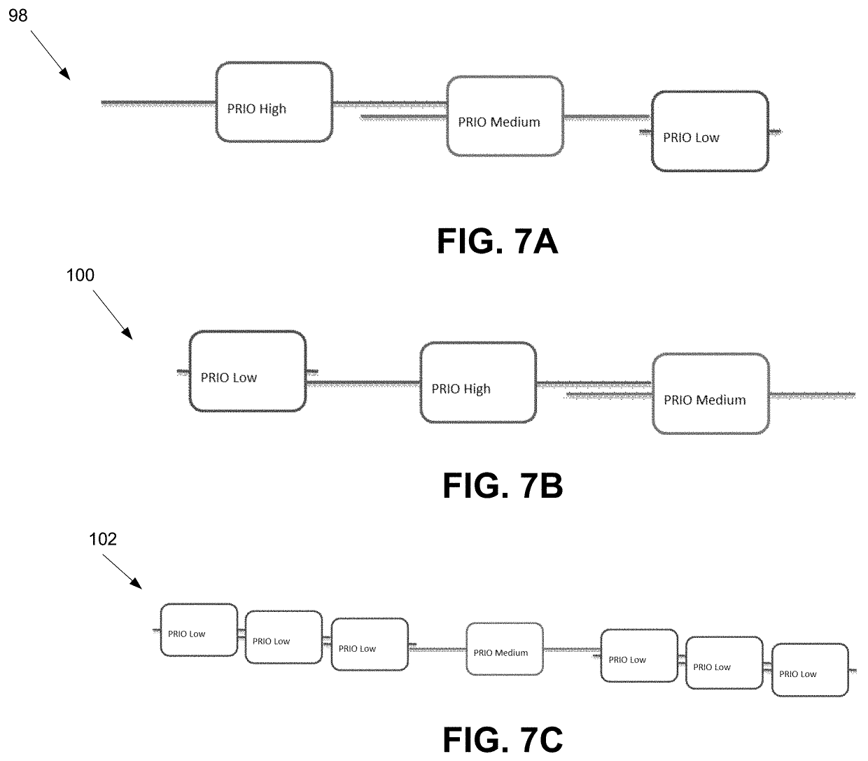

Having defined the silence periods linked to each message, the silence periods are scheduled (e.g., by the silence period definition and scheduling module 88), as shown by the scheduling diagrams 98, 100, and 102 shown in respective FIGS. 7A, 7B, and 7C. In FIGS. 7A-7C, the delivery of messages occurs temporally and consecutively from left to right, where in FIG. 7A, for instance, a first message of high priority is followed by a medium priority message, which is then followed by a low priority message. The priority of the message may be determined based on the importance of the respective message. As shown by the scheduling diagram 98 of FIG. 7A, the silence periods (shown by the lines extending from each message as explained similarly in association with FIG. 6) between messages use the silence period of the higher priority messages, such that the post-silence period of the high priority message is used instead of the pre-silence period of the medium priority message, and the post-silence period of the medium priority message is used instead of the pre-silence period of the low priority message. In the scheduling diagram 100 of FIG. 7B, the messages are delivered such that the first message is a low priority message, the subsequent message is a high priority message, followed by a medium priority message. Accordingly, the pre-silence period of the high priority message is used instead of the post-silence period of the preceding low priority message, and the post-silence period of the high priority message is used instead of the pre-silence period of the subsequent medium priority message. In the scheduling diagram 102 of FIG. 7C, the message delivery is implemented in the following sequence: low priority, low priority, low priority, medium priority, low priority, low priority, and low priority. In such an instance, the silence periods between the same priority messages are equal (co-extensive), yet the pre-silence period of the medium priority message is used instead of the post-silence period of the preceding low priority message, and the post-silence period of the medium priority message is used instead of the pre-silence period of the subsequently followed low-priority message. In general, for the examples illustrated in diagrams 98-102 in FIGS. 7A-7C, the silence values of the higher importance messages have priority, which means, for instance, that if a medium importance message is following a high importance message, the pre-silence of the medium message will be determined by the post-silence of the high importance message. In some embodiments, the priorities may be re-defined from those illustrated in FIGS. 7A-7C. For instance, the importance of the message may be negatively correlated with the silence periods, which may mean that the highest priority message has the smallest duration silence period. For instance, for messaging in an emergency system or application, it is desirable to deliver the most important message (e.g., emergency situation) immediately (e.g., pre-message duration of zero).

In one embodiment, and referring back to operations of the application software 30, the importance value (e.g., determined by the classifier module 86) may be a number between 0 and 1, where 0 indicates non-important messages, and 1 indicates very important messages. The message importance factor can be set by a coach, or can be learned from the user data. The message importance factor can be a static value, or a dynamically changing value (e.g., changing based on current user activity). There are a variety of ways the classifier module 86 may set the importance value for the message (e.g., for the card), one or more of which may be used. The importance value may be set by a coach that makes the plan (e.g., health plan). The importance value may be learned from user data. For example, the type of cards (e.g., educational, interactive, coaching, short, long) that the user reacts to or views (e.g., faster and/or more obediently) may be given higher importance values. Another example is that, depending on the user goals or progress, the importance value can change. For a weight loss program, exercise cards may have higher importance at the beginning, and once the user reaches a certain average activity level, then the eating cards can start becoming more important. In some instances, the importance value may be learned using data mining, including by analyzing the data of all available users. Simulations may be used to simulate user behavior in response to cards, and card importance values can be learned from these simulations. Family and environmental factors (e.g., user meta data) can be used while determining the importance value. Questionnaires may be used to learn the user preferences, and the importance of card types can be set according to the user preferences. In some embodiments, the importance values are dynamic values, and with the user changing behavior and progressing in the program, the importance values may change as well.

In one embodiment, the silence period(s) around a message are computed (e.g., by the silence period definition and scheduling module 88) based on the following equations:

Pre-silence=max(pre-silence-base×importance factor, min-pre-silence) (Eqn. 1A)

Post-silence=max(post-silence-base×importance factor, min-post-silence) (Eqn. 1 B)

In one embodiment, pre-silence-base and post-silence-base are the maximum allowed silences (e.g., 24 hours). Min-pre-silence and min-post-silence are allowed minimum silences (e.g., 10 minutes).

An example method implemented by the application software 30 is described below, but first, a discussion of a few parameter terms, definitions and/or constraints follows.

Card triggering time: The time a card is triggered by a coaching program to be delivered to the user.

Card delivery time: The time a card is delivered to the user's device.

Card viewing time: The time a user views the card. If the card is viewed in the coaching app, the exact viewing time and duration of viewing can be determined. If the message is delivered as a push message, and the user action (e.g., closing) of the push message is tracked, the viewing time can be approximated. If the card viewing time cannot be monitored as described above, then it can be estimated taking into account user behavior information.

If it is known that a record of the first step of the day is T0, and the average record of the last step of the day is T1 (e.g., from historical data), and if the card is delivered in T0-T1 timeframe, then it is estimated that the card viewing time is equal to the card delivery time.

If the card delivery time is outside the average (e.g., calculated from historical data) T0-T1 time frame, then card viewing time is considered to be equal to the T0. For example, for a 7:00-23:00 time frame, if the card is delivered at 6:00, then the card viewing time is estimated to be 7:00 in the same day. If the card delivery time is at 24:00, then the card viewing time is estimated to be 7:00, the next day.

Pre-message silence parameter (PreS): corresponding to silence preceding the message. Calculated as shown in Eqn. 1A.

Post-message silence parameter (PostS): corresponding to the silence that will follow the message. Calculated as in Eqn. 1B.

Actual pre-message silence (aPreS): the actual silence preceding the message, calculated by taking into account the times delivered messages are (actually) viewed by the user.

Actual post-message silence (aPostS): the actual silence after the user has viewed the message. Calculated by taking into account the actual time the user views the current message and the following message.

Current card: the card triggered by the coaching program.

Previous card: the card last viewed by the user.

Assume for illustration a coaching program comprising coaching messages carried out using 5 cards, where each coaching message has an importance value (where some coaching messages are more important than others), each importance value ranging between 0 and 1 inclusive, where 0 indicates a non-important message and 1 indicates a very important message. The following importance determinations are illustrative in Table 1 below:

TABLE 1

| Message | Importance |
|---|---|
| Card 1 | 0.7 |
| Card 2 | 0.9 |
| Card 3 | 0.4 |
| Card 4 | 0.8 |
| Card 5 | 0.3 |

As is explained further below, the silence periods are based on the importance values in one embodiment, or similarly, linked to a feature or parameter of each message, and hence dependent on the content (and/or the context of delivery) of each message. In contrast, in a traditional coaching program, the delivery time of messages happens as soon as the messages are triggered by the system. In other words, there are neither pre- nor post-silences linked to or adapted according to the message. Stated otherwise, in traditional coaching programs, the silence periods are independent from the message content or context. Accordingly, in one embodiment, the application software 30 adjusts the silences (silence periods) according to an importance of the coaching message. For instance:

if importance >=0.5 then message=high importance message;
If importance <0.5 then message = low importance message;

The pre- and post-silence period (parameters) may be set as follows:

For high importance message PreS = 24 hours, and PostS = 48 hours;
For low importance message PreS = 10 minutes, and PostS = 10 minutes.

Note that these values for importance, PreS, PostS are merely used for illustration, and that in some embodiments, other values may be used. In one embodiment, longer silence periods are inserted before or after the messages with higher importance, and each message has two silence parameters: PreS and PostS. Having these silence parameter, application software 30 delivers the messages to the user so that for each message that the user receives, the duration of the actual pre- and post-silences (i.e., aPreS and aPostS, respectively) are not less that the set pre-(PreS) and post- (PostS) silences. Note that reference here to “actual silences” refers to the silences experienced or perceived by the user (note that references below to “viewed” or the like are illustrative of one implementation, and contemplate non-visual experiences or perceptions by the user for some embodiments).

Note that in some embodiments, PreS and PostS may be based on the original coaching program silences. For instance, assuming an observation or estimation of consecutive silence periods 0 through 6, the silence periods may be computed as follows:

```
    For high importance message PreS = mean(silence0-6)*2, PostS =
PreS*2.
    For low importance messages PreS = PostS = mean(silence0-6).
```

One embodiment of an algorithm implemented by the application software 30 is described below in the form of pseudo code, with the understanding that some embodiments may add to or omit certain functions. In a preliminary step (Step 0):

CurrentCard = [ ];
previousCard = card1;

In a first step (Step 1):

Coaching program triggers a new card: card2

CurrentCard = card2;
Card2_triggerTime = T0.
Card2_deliveryTime = [ ]; (not delivered to the user device yet)
Card2_viewingTime = [ ]; (not viewed by the user yet)

In a second step (Step 2):

Calculate the card2_PreS and card2_PostS values. For instance, the card may be classified as having a high or low importance, and then the PreS and postS are calculated as described above.

In a third step (Step 3):

Retrieve the last card information that the user has viewed (or received if the viewing information is not available). This is card 1 (i.e., previous card). So card1_PostS is retrieved.

In a fourth step (Step 4):

Calculate the actual pre- and post silence values for card 2 and card 1 respectively.

For instance, in one embodiment:

card2_aPreS = Current Time − Time user viewed card 1
card1_aPostS = card2_aPreS.

In general, the above card2_aPreS and card1_aPostS can be formulated as follows:

currentCard_aPreS = current time − time user viewed previous card
previousCard_aPostS = currentCard_aPreS

In a fifth step (Step 5):

Delay delivery of the card 2 to the user until the following conditions are satisfied:

card1_aPostS>=card1_PostS & card2_aPreS>=card2_PreS

In general, the above conditions may be formulated as follows:

previousCard_aPostS>=previousCard_PostS
currentCard_aPreS>=currentCard_PreS

Note that in one embodiment, the silence periods may be added to the card in the cloud (e.g., via the computing device 66 executing the application software 30C, and in particular, the delivery module 90) by delaying the delivery of the card to the user's device (e.g., the wearable device 12 and/or the electronics device 14) until the required conditions are satisfied. In other words, card_TriggerTime<=Card_DeliveryTime. In some embodiments, the delaying of the card may happen in the user's device (e.g., the wearable device 12 and/or the electronics device 14). For instance, as soon as a card becomes triggered by the coaching plan, the message may be silently (transparently) sent to the user's device, where the user is not notified of its availability. The card is stored in the memory of the device until the required silence conditions are satisfied. Once the conditions are satisfied, the user is notified that there is a card available.

In a sixth step (Step 6):

When conditions in step 5 are satisfied, deliver the card to the user.

PreviousCard = CurrentCard (i.e. Card 2)
CurrentCard = [ ]; (empty)

In a final step (Step 7), the algorithm returns to step 1.

Note that variations to the above are contemplated to be within the scope of the disclosure, with the application software 30 handling these variations. For instance, if the user never viewed card 1, then the viewing time of the card last viewed by the user is taken into account. If there is no such card, then card 2 may be delivered as soon as it is triggered. As another example, if the user takes too long to view card 2, the silence values are taken into account and adapted accordingly, for example by considering historical aPreS and aPostS values. In this case, it can mean that the user does not want to view the cards frequently and therefore the cards can be delivered with lesser frequency (e.g., longer silence between messages). Another example variation is where a new card 3 is triggered before the user has viewed card 2. Since one reason behind employing silences is to give sufficient room/time to the user to reflect on the messages, one approach is to take into account the time of the last viewed card and make the decision accordingly. In this case, set previousCard=card 1, and CurrentCard=card 3 and continue from step 1. If the cards are sequentially connected, and if for purposes of the program it is important that the cards be viewed in a particular order, then card 2 can be resent to the user if it is not viewed within certain time of its delivery time. For example, if currentTime−cardDeliveryTime>function (card_PreS, card_PostS) and card_ViewingTime=[ ], then the program triggers and delivers card 2 again. In case of the sequential program, if the queue of cards that need to be delivered to the user grows too much due to the user not viewing the previous cards on time (while the program continues to trigger new cards), one approach is to take this into account and adapt the card triggering frequency of the program, and adapt the card silence periods accordingly. Alternatively, the low importance cards can be ignored to empty the queue, or only the last important card can be taken into account, and all previous cards can be ignored.

As explained above, silence periods may be defined based on one or more features of each message, including based on importance, priority, frequency, type, length, delivery channel, context, delivery time, among other features/parameters of the message/card. A simple formula (Eqns. 1A, 1B) is described above for computing silence periods. However, in some embodiments, the equations may be adapted to take into account different, low-level features of each card. And, each card feature may have its own scaling factors, and the final silence values may be determined as a function of all of these scaling factors. In other words, each low level feature may have its own individual importance factor, as shown in table 2 below.

TABLE 2

| | Importance factor for |
|---|---|
| Card low level feature | each card feature |
| Audio message | 0.5 |
| Text message | 0.9 |
| Education content | 0.3 |
| Feedback on progress content | 0.6 |
| Sentiment derived using natural language | 0.9 |
| processing | |

Similar to card (low level) features as shown in the table above, user environmental (e.g., location, temperature, noise, number or people around), state (e.g. high heart rate (HR), stressed, etc.), or activity type (e.g. running, watching TV, sleeping) features may be taken into account while calculating the importance value of a triggered card. In some embodiments, as described above, device and/or network related features may be taken into account (e.g., if the memory of the device is full, if multiple applications want to communicate with the user, if multiple devices in the network want to communicate with the device or the user, etc.). One or a combination of different strategies may be employed. For instance, cards suited to the current user features may be assigned higher or lower importance parameters, so that the cards are delivered quicker or delayed more. As another example, cards preventing the user from engaging in a habit breaking activity may be assigned higher or lower importance value so that they are delivered quicker or later to the users. The implementation can be similar to the example given above. Each low level user or environmental feature can have an importance factor, and the final card importance factors can be calculated by taking these individual factors into account (e.g. multiplying), as illustrated by example in table 3 below:

TABLE 3

| Card features | Card Importance |
|---|---|
| Card 1: educational, audio card | 0.3 × 0.5 = 0.15 |
| Card 2: feedback, text card | 0.9 × 0.6 = 0.54 |
| Card 3: feedback, audio card | 0.6 × 0.5 = 0.3 |
| Card 4: educational, text + audio card | 0.3 × 0.5 × 0.9 = 0.135 |

Note that the example above is merely illustrative of one example implementation, and that in some embodiments, importance values (and/or other features/parameters) may be computed according to different methods. For instance, a mathematical function of all level features may be used. As another example method, a data mining approach (e.g., using neural networks) may be used, where the importance values are learned from a training data set. The training data set may comprise real (e.g., historical) data and/or simulated (e.g., synthetic) data. Yet another example method comprises a combination of both mathematical and data-based approaches. Note that, though the description above illustrates the use of importance categories (e.g., high or low), in some embodiments, absolute importance values may be used. In this case, PreS and PostS may be calculated by multiplying the importance value by a preset value. For example: PreS=PostS=card_importance*24 hours.

In the previous sections, the use of user data and analysis thereof to guide setting the importance value is explained. In some embodiments, one or more of the following additional features may be monitored (autonomously) and used. For instance, to set the importance value, one or more of the following (example) parameters may be taken into account: (a) the response of the user to the similar types of messages (e.g., to determine similar type messages, messages can be clustered in terms of their low level features); (b) has the user opened and viewed the delivered card; (c) how long did the user spend reading the message; (d) did the user delete the message after reading it; (e) did the user re-open and re-read the message more than once; (f) if it is an informative message with a link, did the user click on the link. How long did he spend at the link location; (g) if it is an actionable message: did the user act upon the message; (h) the distribution of coaching messages in time; and (i) the importance value of the following and/or preceding messages: e.g., analyzing the messages in pairs, or triplets.

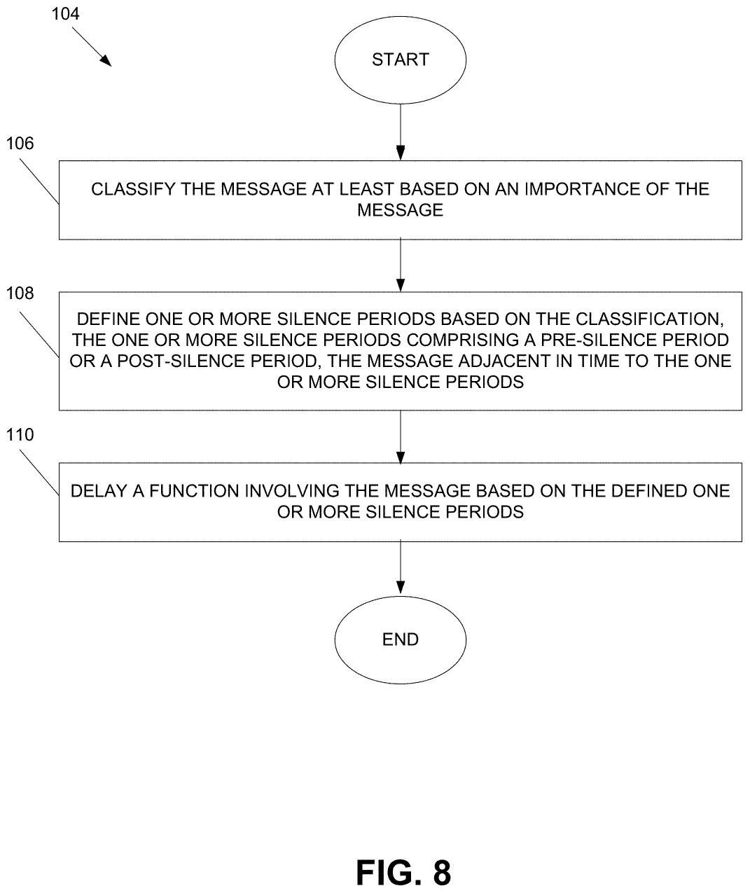

Having described certain functionality of the application software 30 illustrated in FIG. 5, it should be appreciated that one embodiment of an example adaptive messaging method, depicted in FIG. 8 and denoted as method 104, which is shown bounded by start and end, comprises classifying a message at least based on an importance of the message; defining one or more silence periods based on the classification, the one or more silence periods comprising at least a pre-silence period or a post-silence period, the message adjacent in time to the one or more silence periods; and delaying a function involving the message based on the defined one or more silence periods. In one embodiment, the delaying of the function comprises either delaying delivery of the first message relative to the triggering or causing a delay in notification of the first message after the first message is delivered. In some embodiments, the delaying of the function comprises delaying a notification, implementation, or actuation of the message. For instance, a message may be intended to be used to change the specification of a device or algorithm automatically, without user intervention. As an illustration, a message may automatically alter the setting of a phone (e.g., put the device in night mode), and this message is intended to go into effect at a certain time (e.g., 10 pm, or if the triggering of the message is made at 9:59:59, the silence period is 1 second in duration).

Any process descriptions or blocks in flow diagrams should be understood as representing modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For instance, in some embodiments, messages may have several importance values, such as an importance value set by the coach, and an importance value learned from user historical data. Both values may be combined (e.g., multiplied) to get a single importance value per message. Alternatively, depending on the user preferences, time of the day, location, user state, etc., an importance value to be used may be selected. As another example, like a good speaker, it may not be a good idea to have all the important messages grouped temporally close to each other. Instead if the important messages are interspaced in the presentation, that may be more effective. Having the importance values of messages defined, the coaching program messages can be selected so that there is a good balance in how the high and low importance messages are distributed. For example, there can be hard rules implemented requiring at least two low importance messages before a high importance message. In other words, the importance values of the messages can guide the coach in how the program should be constructed. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. An apparatus, comprising:
a memory comprising instructions; and
one or more processors configured to execute the instructions to:
classify a message at least based on an importance of the message;
define one or more silence periods based on the classification, the one or more silence periods comprising a pre-silence period or a post-silence period, the message adjacent in time to the one or more silence periods; and
delay a function involving the message based on the defined one or more silence periods.

2. The apparatus of claim 1, wherein the one or more processors are further configured to execute the instructions to classify the message based on one or more additional parameters, the one or more additional parameters comprising a priority of the message, a frequency of message delivery, a content of the message, a type of the message, or prior silence periods.

3. The apparatus of claim 1, wherein the one or more processors are further configured to execute the instructions to classify the message based on one or more additional parameters, the one or more additional parameters comprising a delivery time of the message, a delivery channel of the message, a historical distribution of messages, a current user state, an observed reaction to prior messages, a format of the message, or environmental factors.

4. The apparatus of claim 1, wherein the one or more processors are further configured to execute the instructions to classify the message based on one or more additional parameters, the one or more additional parameters comprising a response of a user to a similar type of message, whether the user has opened and viewed a card for delivering the similar type of message, how long the user read the similar type of message, whether the user deleted the similar type of message after reading it, whether the user re-opened and re-read the similar type of message, whether the user selected a link located within the similar type of message, how long the user spent at a location corresponding to the link, or whether the user acted on the similar type of message.

5. The apparatus of claim 1, wherein the one or more processors are further configured to execute the instructions to classify by setting an importance value corresponding to the importance, the importance values set according to any one of a range of values.

6. The apparatus of claim 5, wherein the one or more processors are further configured to execute the instructions to set the importance value based on one or any combination of third party input or learning from user data.

7. The apparatus of claim 6, wherein the learning is based on one or any combination of data mining a population of users, simulations of user behavior in response to prior messages, analyzing historical user reaction to the prior messages, analyzing family history, analyzing environmental factors, or ascertaining user preferences from questionnaires.

8. The apparatus of claim 6, wherein the one or more processors are further configured to execute the instructions to set the importance value based on one or a combination of user goals or progression towards the user goals.

9. The apparatus of claim 5, wherein the one or more processors are further configured to execute the instructions to define the one or more silence periods by:
providing a respective duration to the one or more silence periods based on the importance value;
determining an actual pre-silence period for the message and a post-silence period for a prior message; and
evaluating one or more conditions related to the respective duration of the one or more silence periods and the determined actual pre-silence and post-silence periods, wherein the delayed function comprises a delayed delivery or delayed notification, wherein the delayed function or delayed delivery is based on at least meeting the respective durations.

10. The apparatus of claim 5, wherein the importance value is based on a combination of plural importance values, wherein a first importance value of the plural importance values corresponds to an importance to a third party and a second importance value of the plural importance values corresponds to an importance to a user.

11. The apparatus of claim 1, wherein the one or more processors are further configured to execute the instructions to classify a card delivering the message and defining the one or more silence periods further based on the classification of the card, wherein each delay is further based on the one or more silence periods defined by the classification of the message and the classification of the card.

12. The apparatus of claim 11, wherein the one or more processors are further configured to execute the instructions to classify the card by determining an importance value for respective one or more features of the card, the classification of the card comprising a function of the respective one or more importance values.

13. The apparatus of claim 12, wherein the one or more processors are further configured to execute the instructions to determine the importance value based on one or more contexts, wherein the contexts include an environment of a user, state of the user, or activity type of the user.

14. The apparatus of claim 1, wherein the one or more processors are further configured to execute the instructions to repeat the classify, define, and delays for plural messages, wherein the one or more processors are further configured to execute the instructions to vary an order of the plural messages based on the respective importance.

15. The apparatus of claim 1, wherein the one or more processors are further configured to execute the instructions to define plural silence periods based on the classification, the plural silence periods comprising the pre-silence period and the post-silence period.

16. The apparatus of claim 1, wherein the one or more processors are further configured to execute the instructions to delay the function by delaying delivery of the first message relative to a triggering, causing a delay in notification of the first message after the first message is delivered, or delaying the notification, implementation, or actuation of the message.

17. The apparatus of claim 1, wherein the one or more processors are further configured to execute the instructions to modify or replace the message prior to delivery based on updated information.

18. A method, comprising:
classifying a message at least based on an importance of the message;
defining one or more silence periods based on the classification, the one or more silence periods comprising a pre-silence period or a post-silence period, the message adjacent in time to the one or more silence periods; and
delaying a function involving the message based on the defined one or more silence periods.

19. The method of claim 18, further comprising denying delivery of any additional messages during the one or more silence periods, the denial implemented at a local device or network level.

20. A non-transitory, computer readable medium comprising instructions that, when executed by one or more processors, causes the one or more processors to:
classify a message at least based on an importance of the message;
define one or more silence periods based on the classification, the one or more silence periods comprising a pre-silence period or a post-silence period, the message adjacent in time to the one or more silence periods; and
delay a function involving the message based on the defined one or more silence periods.
